(19) United States
(12) Patent Application Publication
(10) Pub. No.: US 2006/0264778 A1
(43) Pub. Date: Nov. 23, 2006

(54) Verification method and system for medical treatment

(75) Inventors: Bernard C.B. Lim, Oakville (CA); Taras Worona, Etobicoke (CA); Hao Chen, Mississauga (CA); Kathleen Chancellor-Maddison, Hamilton (CA); Davis A.R. Kanbergs, Milton (CA)

Correspondence Address: BUCHANAN, INGERSOLL & ROONEY PC, POST OFFICE BOX 1404, ALEXANDRIA, VA, 22313-1404, US

(73) Assignee: Vasogen Ireland Limited, Shannon (IE)

(21) Appl. No.: 11/435,980

(22) Filed: May 18, 2006

Related U.S. Application Data
(60) Provisional application No. 60/683,280, filed on May 19, 2005.
(60) Provisional application No. 60/682,969, filed on May 19, 2005.
(60) Provisional application No. 60/683,333, filed on May 19, 2005.

(51) Int. Cl. A61B 5/00

(52) U.S. Cl. 600/576

(57) ABSTRACT

A system for the collection, treatment and delivery of an autologous blood sample, comprising a first syringe for drawing an untreated blood sample from a patient, a blood sample treatment chamber having a chamber inlet for receiving untreated blood from the first syringe and chamber outlet for passage of treated blood to a second syringe coupled thereto. The second syringe includes a releasable lock means for allowing discharge of the treated blood to the patient in response to a release signal. The release signal is issued following a positive outcome from a verification process dependent upon temporal data from certain events in the collection, treatment and delivery of the blood sample, and identity data of the patient and the second syringe with the treated blood.

### CROSS-REFERENCE TO RELATED APPLICATIONS

This application claims the benefit of priority to U.S. Provisional Application Ser. No. 60/682,969, filed May 19, 2005, U.S. Provisional Application Ser. No. 60/683,280 filed May 19, 2005, and U.S. Provisional Application Ser. No. 60/683,333, filed May 19, 2005.

### BACKGROUND OF THE INVENTION

1. Field of the Invention

The present invention relates to the management of medical treatments. More specifically it relates to a permission-based fluid dispensing device.

2. Description of the Prior Art

Despite remarkable advances in health care technology and delivery, a large number of patients die or are disabled as a result of medical errors. These errors occur in health care settings, such as hospitals, clinics, nursing homes, urgent care centers, physicians' offices, pharmacies, and the care delivered in the home, and they usually result from systems problems rather than one single action or decision.

For many years, bar code labelling has been the technology of choice in ensuring patient safety. Recently, the Food and Drug Administration (FDA) issued a new rule which requires certain human drug and biological product labels to have bar codes. As such, the bar code for human drug products and biological products (other than blood, blood components, and devices regulated by the Center for Biologics Evaluation and Research) must contain the National Drug Code (NDC) number in a linear barcode. The rule is geared toward reducing the number of medication errors in hospitals and other health care settings by allowing health care professionals to use bar code scanning equipment to verify that the right drug (in the right dose and right route of administration) is being given to the right patient at the right time. The rule also requires the use of machine-readable information on blood and blood component container labels to help reduce medication errors.

However, bar codes require line of sight with a reader in order to be read and they cannot store additional information apart from simple identification data, such as a serial no. or SKU. For example, a bar-coded wristband on a patient is not easy to read if the patient gets it wet or is sleeping on top of the arm bearing the wristband, or when the patient is on an emergency room gurney or operating table; these are instances where mistakes in medication or blood transfusion are most prevalent.

It is an object of the present invention to mitigate or obviate at least one of the above-mentioned disadvantages.

### SUMMARY OF THE INVENTION

In one of its aspects, the present invention provides a system for the collection, treatment and delivery of a blood sample, the system comprising:
an article for association with a patient having a patient identifier;
a first syringe having:
   a first syringe inlet for drawing an untreated blood sample from the patient,
   a first fluid chamber for receiving the untreated blood,
   a first syringe outlet for dispensing the untreated blood sample from the first chamber,
   a first incremental counter for recording temporal data corresponding to untreated blood events related to the collection of blood,
   the first syringe being associated with a first unique identifier correlatable to the patient identifier;
a vessel for processing the blood sample, the vessel having:
   a blood sample processing chamber, the vessel having a chamber inlet; the first syringe outlet being operable to establish a dedicated first fluid coupling with the chamber inlet to dispense the untreated blood sample to the blood sample processing chamber; the vessel having a chamber outlet for dispensing a treated blood sample following treatment to a second syringe,
the second syringe having:
   a second syringe inlet operable to form a dedicated second fluid coupling with the chamber outlet to receive the blood sample from the blood sample treatment chamber;
   a second chamber for receiving the treated blood;
   a second syringe outlet;
   a passage in communication with the second chamber and the second syringe outlet;
   a second incremental counter for recording temporal data corresponding to blood treatment events, treated blood events and delivery events; the second incremental counter being operable independently of the first incremental counter and being non-synchronized with the first incremental counter;
   the second syringe being associated with a second unique identifier, the second unique identifier operatively associated with the first syringe and correlatable to the first unique identifier;
   a releasable lock formed within the passage for operating the second syringe outlet between a plurality of states;
a processor having:
   a comparator for comparing the patient identifier to the first unique identifier to determine the correlation between same; and comparing the second unique identifier to the patient identifier to determine the correlation between same, the comparator issuing an output signal;
   logic for receiving the output signal and the temporal data to determine time delays between the events and for determining whether the time delays are within predefined ranges;
a release signal generator coupled to the logic for issuing a release signal to the releasable lock;
   whereby the release signal is issued upon confirmation of the correlation between the patient identifier and the first unique identifier, and the correlation between the patient identifier and the second unique identifier, and provided that the time delays are within predetermined ranges.

In another of its aspects, the present invention provides identification means for identifying an originating patient of the untreated blood sample, verification means for verifying a match between the originating patient and the treated blood sample, and release signal generating means for generating a release signal in response to a positive verification by the verification means. The identification means and/or the release signal generating means may be located on the second syringe body, or on an external article. The external article may worn, carried, attached or ingested by the patient, such as a pinned or self adhesive label, or a coated object, and the like. Preferably, the external article contains a removable portion containing audit data relating to the patient and/or the treated blood sample. For example, the external article may be conveniently provided as a wristband to be worn by the originating patient.

In yet another of its aspects, the second syringe body may also include a filtered vent outlet in the passage for expelling one or more gas constituents in the treated blood sample.

As a further aspect, the present invention provides a method of monitoring a material sample from a patient, comprising the steps of:
(a) collecting the sample from the patient with a first collection device;
(b) associating the patient with a first signal carrying data representative of the sample;
(c) associating the first collection device with a second signal carrying data representative of the sample;
(d) delivering the sample to a sample treatment chamber;
(e) processing the sample to form a processed sample;
(f) collecting the sample in a second collection device;
(g) associating the second collection device with a third signal carrying data representative of the processed sample;
(h) comparing the data in the first and third signals to link the processed sample with the patient; and thereafter;
(i) associating at least one of the steps (a) to (h) with temporal data;
(j) determining at least one time delay using the temporal data to determine whether the at least one of the steps (a) to (h) occurs within acceptable time limits;
(k) delivering the processed sample to the patient upon a positive outcome from step (h) and step (j); and
(l) assembling an audit record having temporal data collected from step (i), the outcome from step (h) and step (j), and data associated with the sample.

The events related to the collection of untreated blood are tracked by the first incremental counter, while the treatment and post treatment events are tracked by the second incremental counter, such that time delays may be determined from the temporal data Advantageously, these two counters operate independently of one another and do not require to be synchronized with each other, unlike real-time clocks. The counters only operate during the steps (a) to (j) described above, and thus do not require substantial battery power. As such, the battery is sufficient to maintain substantial accuracy of the clock within the time period from steps (a) to (f), and thus the possibility of losing time or decreasing clock accuracy as the battery's power runs down is substantially eliminated.

In yet another of its aspects, the system includes a releasable lock means operable by a solenoid configured to receive the release signal.

In yet another of its aspects, the system includes a releasable lock means operable by a motorized means configured to receive the release signal.

The term “treatment device” used herein below is intended to mean a device used directly or indirectly in the course of a treatment. It may include devices which actually perform a treatment on the patient or a patient-derived sample, or alternatively be an article for performing functions associated with treatments, such as carrying or otherwise transferring the sample to or from a treatment.

### BRIEF DESCRIPTION OF THE DRAWINGS

These and other features of the preferred embodiments of the invention will become more apparent in the following detailed description in which reference is made to the appended drawings wherein:

FIG. 1 is a perspective view of a blood treatment system;

FIG. 2 is a sectional view of a first syringe shown in FIG. 1, taken along line 1-1′;

FIG. 3 is a perspective view of the first syringe of FIG. 1 coupled to a sodium citrate bag;

FIG. 4 is a perspective view of a blood treatment chamber of FIG. 1;

FIG. 5 is a perspective view of a second syringe of FIG. 1;

FIG. 6 is a sectional view of the second syringe of FIG. 5 taken along line 5-5′;

FIG. 7 is another perspective view of the blood treatment chamber carrying the first syringe and the second syringe;

FIG. 8 is a sectional view of the blood treatment chamber of FIG. 7 taken along line 7-7′;

FIG. 9 is a sectional view of the blood treatment chamber of FIG. 7 taken along line 9-9′;

FIG. 10 is an exploded view of an outlet port of the second syringe of FIG. 5;

FIG. 11 is a perspective view of a outlet valve;

FIG. 12 is a sectional view of the outlet valve element of FIG. 10 taken along line 11-11′;

FIG. 13(a) is a perspective view of the a portion of locking mechanism in a locked state;

FIG. 13(b) is a perspective view of the a portion of locking mechanism in an open state;

FIG. 13(c) is a perspective view of the portion of locking mechanism in a permanently locked state;

FIG. 13(d) is a perspective view of the portion of locking mechanism adjacent to the outlet port of FIG. 10, in a permanently locked state;

FIG. 14 is a perspective view of the portion of locking mechanism in a cooperating arrangement with the outlet port;

FIG. 15 is a flowchart outlining a verification protocol of the system of FIG. 1;

FIG. 16 is a flowchart outlining a verification portion protocol of FIG. 15;

FIG. 17 is a detailed perspective view of the blood treatment system;

FIG. 18 is a schematic view of a verification protocol;

FIG. 19 is a perspective view of a wristband as shown in FIG. 1, prior to operation;

FIG. 20 is a perspective view of a wristband as shown in FIG. 1, in operation; and;

FIG. 21 is a perspective view of a wristband as shown in FIG. 1, prior to operation.

### DESCRIPTION OF THE PREFERRED EMBODIMENTS

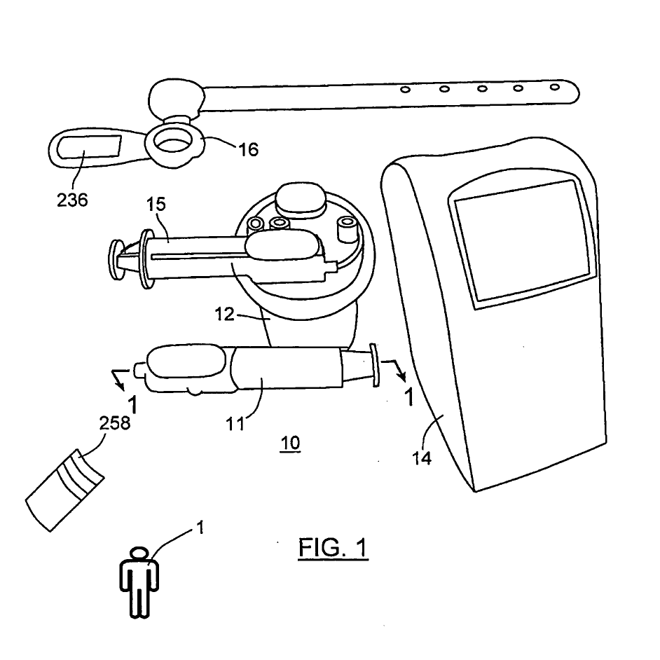

As shown FIG. 1, there is provided a system 10 for the collection, treatment and delivery of an autologous blood sample. The system 10 includes a plurality of entities which are used at different stages during the handling of the blood sample, such as, a first syringe 11 (S1), a sample management unit 12 (SMU), a blood treatment unit 14 (BTU), a second syringe 15 (S2), and a wristband 16 (WB). The first syringe 11 is used to collect an untreated blood sample from an originating patient 17. Following collection of the untreated blood sample, the blood collection syringe 11 is coupled to the sample management unit with the blood delivery syringe 15 already mounted thereon, and the sample management unit is introduced into the blood treatment unit, in which the untreated blood sample is subjected to one or more stressors, such as ozone or ozone/gas mixture, ultra-violet (UV) light and infra-red (IR) energy.

Following treatment, the treated blood sample is delivered to a second syringe 15, from which the treated blood sample is administered to the originating patient 17. At one or more critical stages, the system 10 provides for a verification check, aimed at reducing the possibility of error, and thus ensure that the correct blood sample is returned to the correct originating patient 17. The verification check includes the steps of matching the blood sample, either in its treated or untreated form or both, with the originating patient 17. Typically, the wristband 16, the first syringe 11, the sample management unit 12, the second syringe 15, include identification data associated with the originating patient, the data may include indicia, or may be machine-readable via optical or electro/magnetic means.

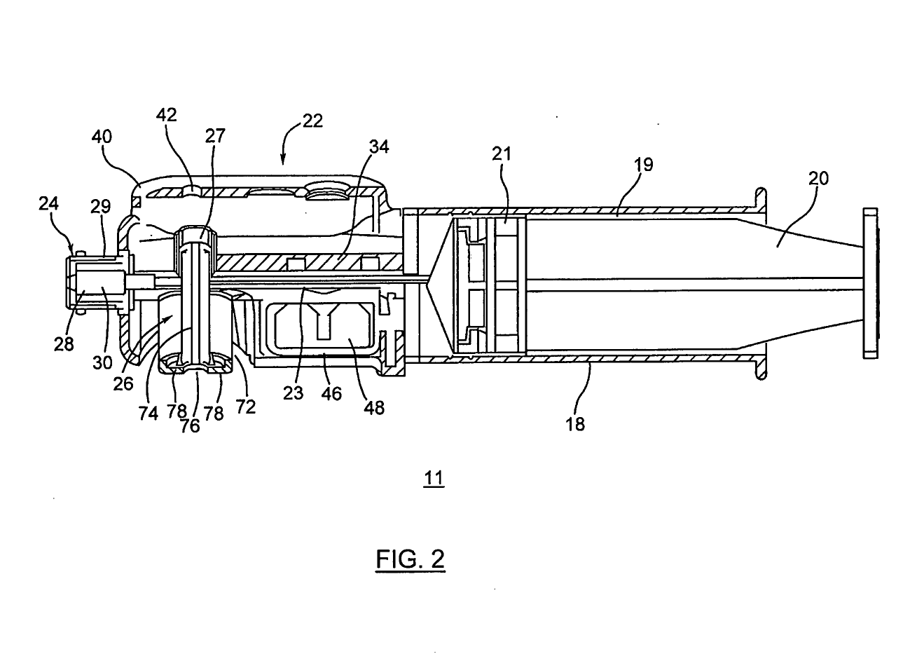

As shown in FIG. 2, the first syringe 11 has a first body portion 18 which provides a cylindrical cavity 19 which in cooperation with a syringe plunger 20 forms a sample receiving chamber 21. The first syringe 11 includes a first channel portion 22 with a channel 23 in communication with the first sample receiving chamber 21, and a first syringe inlet port 24 for ingress of the untreated blood sample from the patient 17. The first channel portion 22 also includes a first syringe outlet port 26 for dispensing the untreated blood sample therefrom to the sample management unit 12. The first syringe outlet port 26 includes a channel 27 in communication with the first sample receiving chamber 21 and channel 24.

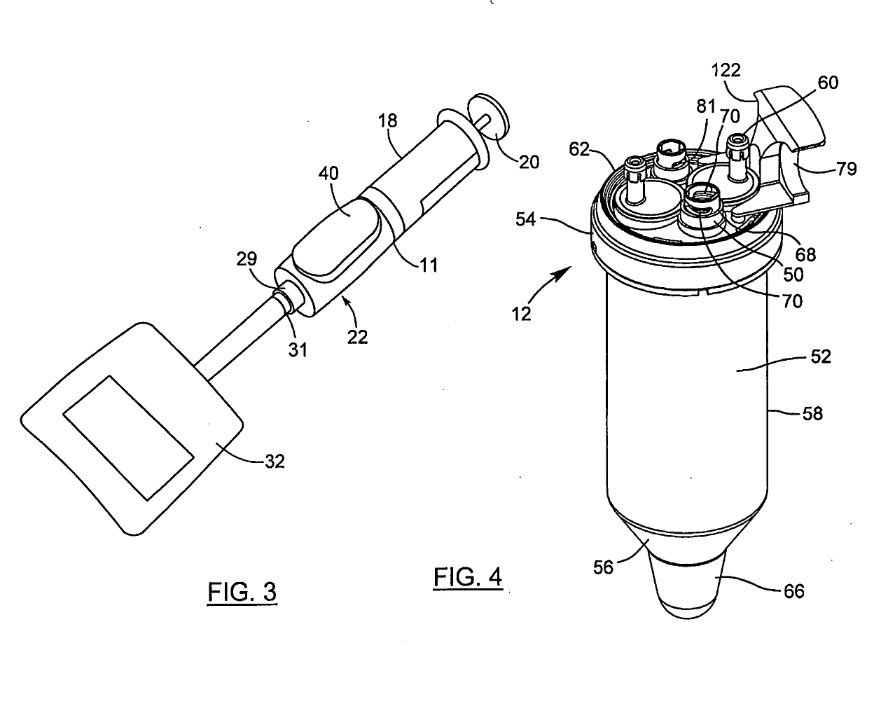

The first syringe inlet port 24 is provided with a first syringe inlet valve means 28 in channel 24 for controlling the flow of blood through the first syringe inlet 24. In this case, the first inlet valve means 28 includes a housing 29 containing a valve 30 arranged to be opened by a complementary valve member 31, located on an external device 32, as shown in FIG. 3. The external device 32 may be a blood collection unit, such as a “butterfly” needle or a sodium citrate bag, and so forth. Extending outwardly from the first syringe outlet port 26 is a pair of bayonet pins 72 for coupling the first syringe 11 to the blood treatment chamber 12. Included within the channel 27 of the first syringe 11 is a valve element 74 biased to a closed position against a valve seat 76 on an end cap 78 which forms the outer end of the first syringe outlet port 26.

Within the first channel portion 22, is a printed circuit board (PCB) 34 having circuitry for transmitting and receiving data related to the syringe and/or its contents, or a patient 17, such as identification data, SKU, serial no., manufacturing date, expiry date, fluid data, health facility data, health practitioner data, medication data, and so forth. The circuitry includes, but is not limited to, a transmitter, a receiver, logic means or processor, a computer readable memory for data storage, a timing circuit, an antenna and a power source. In the preferred embodiment, the circuitry also includes an RFID reader/writer for reading RFID tags associated with other entities within the treatment system. The RFID reader/writer is also coupled to other elements of the circuitry to perform at least one verification check, and other functions. Also coupled to the PCB 34 are input/output devices such as a display, an LED 36, a speaker, and a switch, such as pullout tab 38. The first channel portion 22 also includes a cover 40 with a bore 42 contiguous with an opening 44 of the first outlet port 26. The first syringe 11 also includes a compartment 46 for housing a power supply unit 48 to provide electrical power to the PCB 34 and the input/output devices. The power supply unit 48 typically comprises one or more batteries which may be removed following the single use of the first syringe 11, in order to enable use in another device or allow for proper recycling in compliance with current environmental regulations. In order to facilitate easy battery installation or removal, the batteries 48 may be placed on a tray which is slidably received by the compartment 46.

As shown in FIG. 4, the sample management unit 12 is a vessel 49 having an open top portion 51, a closed bottom portion 56 and a rigid walled portion 58 therebetween, and a cover portion 54 to define a cylindrical treatment cavity 52, or treatment chamber. The cover portion 54 has a chamber inlet 50 to form a dedicated first fluid coupling with the first syringe outlet port 26, such that the untreated blood sample may be dispensed into the treatment cavity 52 of the blood sample treatment chamber 12. The cover portion 54 also has a gas inlet port 60 for delivery of ozone to treat a blood sample, a gas outlet port 62 for the discharge of the ozone, and other gases. The bottom portion 56 has a bowl 66 to receive the blood sample during treatment.

In the course of the treatment of the blood sample, the treatment cavity 52 is subjected to stressors, such as, UV A, B and C radiation, infrared radiation and ozone is bubbled through the blood sample. As such, the walled portion 58 and the bowl 66 are made from appropriate materials capable of transmitting such radiation, such as low density polyethylene (LDPE) containing a small amount (about 5%) of ethylene vinyl acetate. The chamber inlet 50 has a female collar portion 68 with a pair of helically oriented passages or grooves 70 extending through its wall, or in its wall, to receive the pair of corresponding bayonet pins 72 of the first syringe outlet port 26. In operation, the first syringe 11 is rotated to urge the bayonet pins 72 along the helical passages 70 and downwardly into the female collar portion 68 until the valve element 74 abuts the valve-actuating element 80. Subsequently, the valve element 74 is displaced by the valve-actuating element 80 from its closed position against the valve seat 76 to open the fluid coupling. Once fully engaged within the chamber inlet 50, the first syringe 11 is supported in place by a saddle member 79, which minimizes motion of the first syringe about the chamber inlet 50.

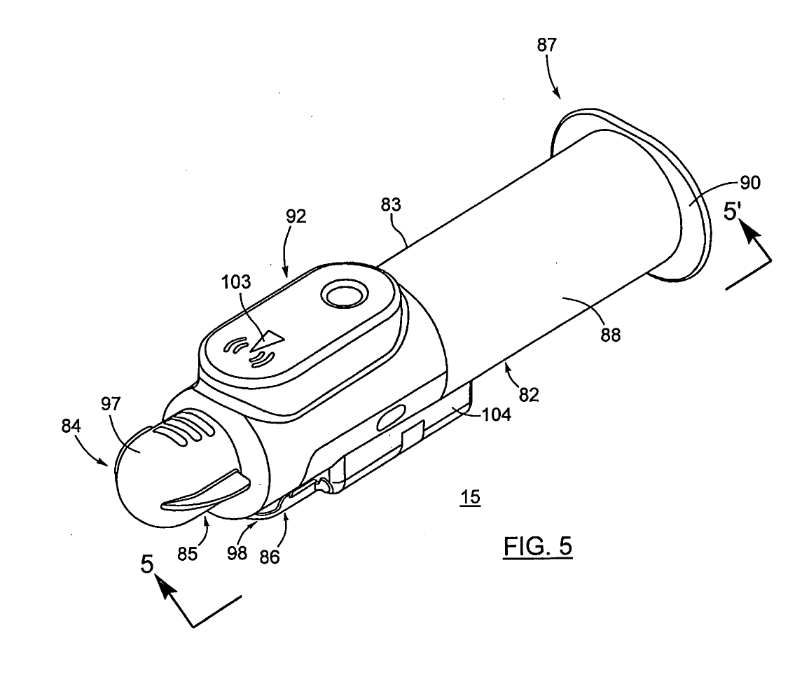

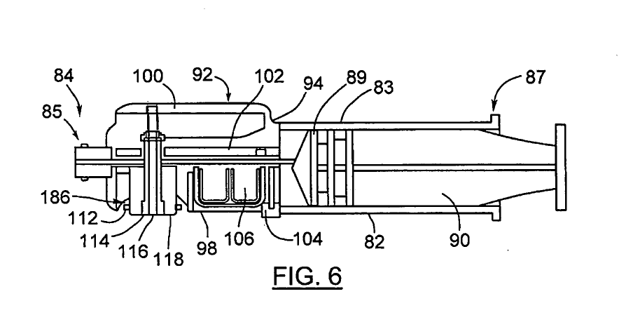

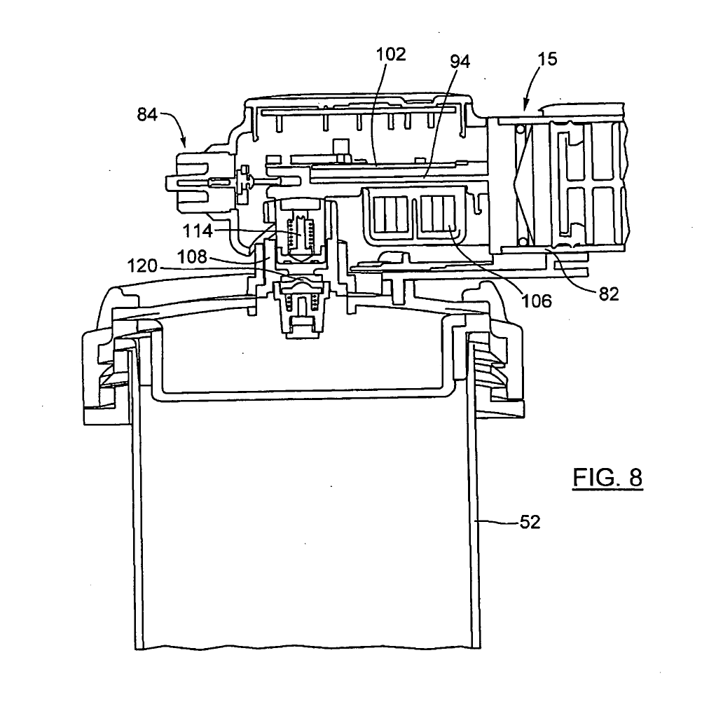

The cover portion 54 has a chamber outlet 81 to form a dedicated second fluid coupling with the second syringe 15, as shown in FIG. 8. The second syringe 15, shown in more detail in FIG. 5 and FIG. 6, has a second body portion 82 having a barrel 83 with a proximal end 84, at which is disposed a second inlet port 85, a second outlet port 86; and a distal end 87 with a cylindrical wall 88 therebetween to define a second sample receiving chamber 89. The second inlet port 85 is disposed at an angle to the second outlet port 86, and intermediate the second sample receiving chamber 89 and the second outlet port 86. A plunger 90 is slidably disposed at the distal end 87 and is in tight fluid engagement with the cylindrical wall 88. The plunger 90 serves to draw fluid into the second sample receiving chamber 89 and urge the fluid therefrom. The second syringe 15 also includes a second channel portion 92 with a channel 94 in communication with the second sample receiving chamber 89 and the second outlet port 86, and a channel 96 in communication with the second inlet port 85 and the second sample receiving chamber 89 via a portion of the channel 94. In order to prevent large particulate from entering the second outlet port 86, a second end cap 97 is removably attached thereto, while the second inlet port 85 includes a slidable cap 98 to prevent contamination prior to use with the blood treatment unit 14. The treated blood sample is dispensed from the second syringe 15 to the originating patient 17 via the second syringe outlet port 86 operable between an open position and a closed position by a releasable lock means 100, as will be described below.

Similar to the first syringe 11, within the second channel portion 92 is a printed circuit board (PCB) 102 having circuitry for transmitting, receiving and storing data related to the syringe and/or its contents or the originating patient 17, such as identification data, SKU, serial no., manufacturing date, expiry date, fluid data, health facility data, health practitioner data, medication data, and so forth. The circuitry includes RFID reader/writer functionality for reading RFID tags associated with other entities within the treatment system. The RFID reader/writer is also coupled to other elements of the circuitry to perform at least one verification check, and other functions As such, the circuitry includes, but is not limited to, a transmitter, a receiver, logic means or processor, a computer readable medium for data storage, a timing circuit, an antenna and a power source. Also coupled to the PCB 102 are input/output devices such as a display, LED 103, a speaker or a button. In addition, the PCB 102 also includes circuitry for controlling the operation of the releasable lock means 100. A compartment 104 houses a power supply unit 106 comprising one or more batteries, and a power circuit resident on the PCB 102 for regulating the power therein and input/output devices. The batteries 106 may be removed after the single use of the second syringe 15, in order to enable use in another device or allow for proper recycling in compliance with current environmental regulations. In order to facilitate easy battery installation or removal, the batteries 106 may be placed on a tray which is slidably received by the battery compartment 104.

The syringe 10 is typically maintained in a low power state, when not in use, to conserve battery energy. However, when the sample management unit 12 is introduced into the blood treatment unit, the syringe 15 is placed into an operating state from the lower power state. Such a transition may be effected via a mechanical switch which is closed before insertion of the sample management unit into the blood treatment unit, or the switch is closed by the blood treatment unit following insertion of the sample management unit into the blood treatment unit. Other ways include an electronic switch actuable by an RF signal or a DC signal from the blood treatment unit, or a DC magnetic reed relay enabled by a magnet in the blood treatment unit.

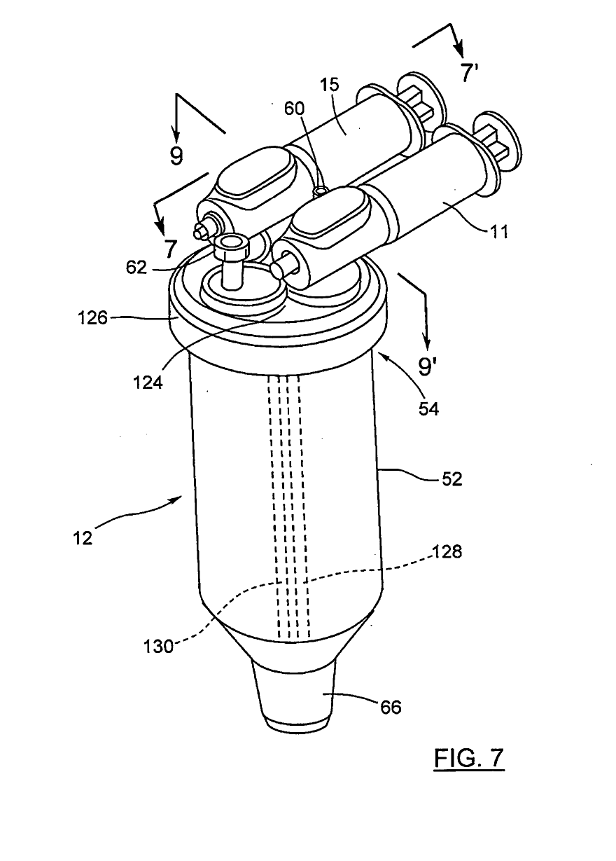

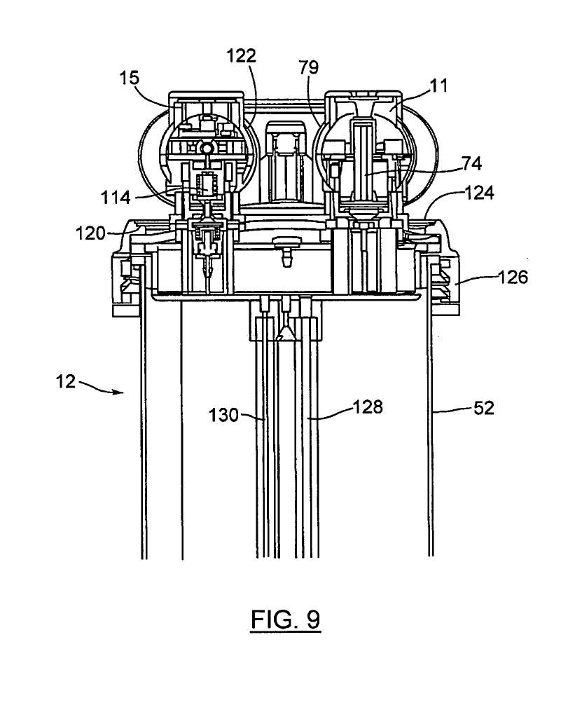

As shown in FIGS. 4 and 7 to 9, the chamber outlet 81 has a female collar portion 108 with a pair of helically oriented passages or grooves 110 extending through or in its wall to engage a corresponding one or more pins 112 extending outwardly from the second syringe inlet port 85. Similarly, a valve element 114 is located in the channel 96 and biased to a closed position against a valve seat 116 on an end cap 118 forming the outer end of the second syringe outlet 96. The valve element 114 is also aligned for abutment with a valve actuating element 120 which is positioned in the chamber outlet 81. The valve actuating element 120 is thus operable to displace the valve element 114 from its closed position against the valve seat 116 to open the second fluid coupling. The cover portion 54 is also provided with a saddle member 122 for supporting the second syringe 15 when it is in a fully engaged position with chamber outlet 81.

The cover portion 54 has a top cap 124 and a cap lock 126 bonded, welded or otherwise fixed thereto. The cap lock 126 latches on an upper periphery of the bottom portion 56. The chamber inlet 50 and the chamber outlet 81 are each in fluid communication with the inner treatment cavity 52 by way of conduits 128, 130 extending below the valve actuating elements 80, 120 respectively.

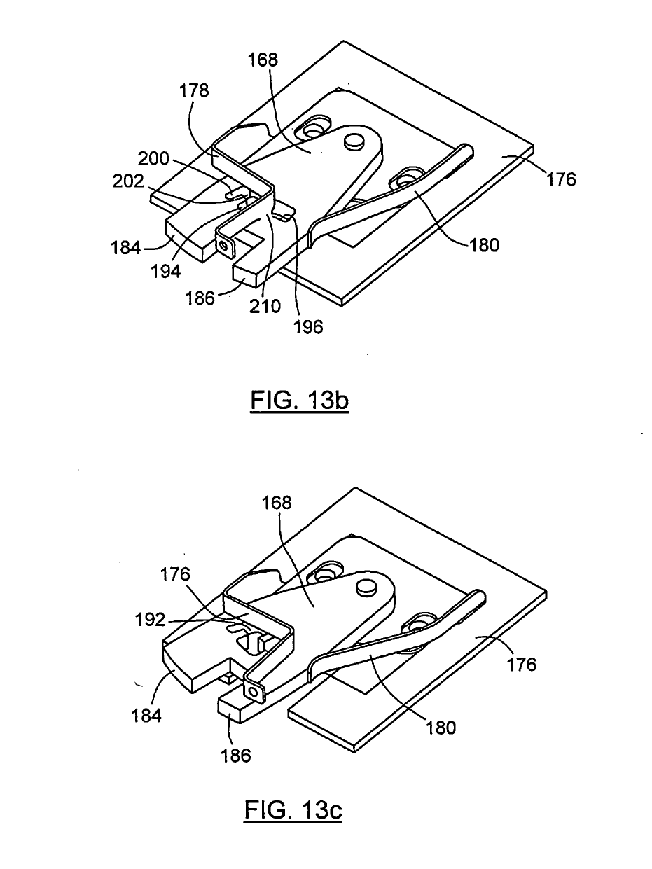

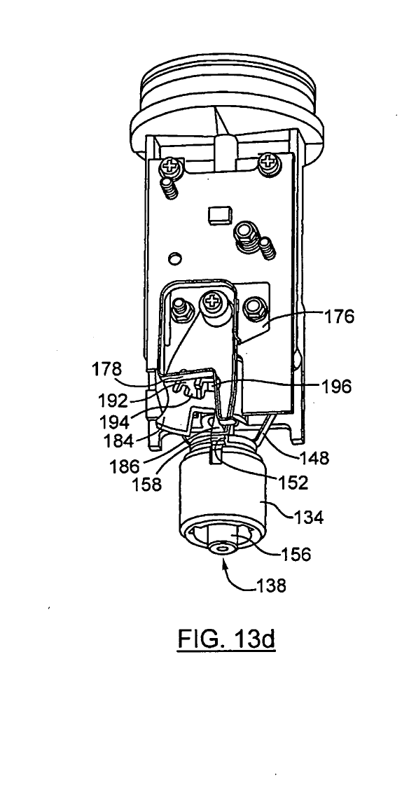

As shown in FIGS. 6, 10, 11 and 12, the second syringe body portion 84 has a cylindrical cavity which in cooperation with the plunger 90 provides a second sample receiving chamber 89. The passage 94 of the blood sample transfer portion 92 has a second access location 132 for fluid communication with the second syringe outlet port 86. The second syringe outlet port 86 and the blood transfer portion 92 are further provided with the releasable lock means shown generally at 100 for forming a locked third fluid coupling between the second access location 132 and the second syringe outlet port 86. As will be described, the releasable lock means 100 is operable in response to a release signal to release the third fluid coupling, as shown in FIGS. 13(a) to 13(d). With the releasable lock means unlocked, the second syringe outlet port 86 is operable to form a fourth fluid coupling with a fluid fitting on a common blood sample delivery unit with a complementary LUER 31 or similar fitting, such as the needle 32.

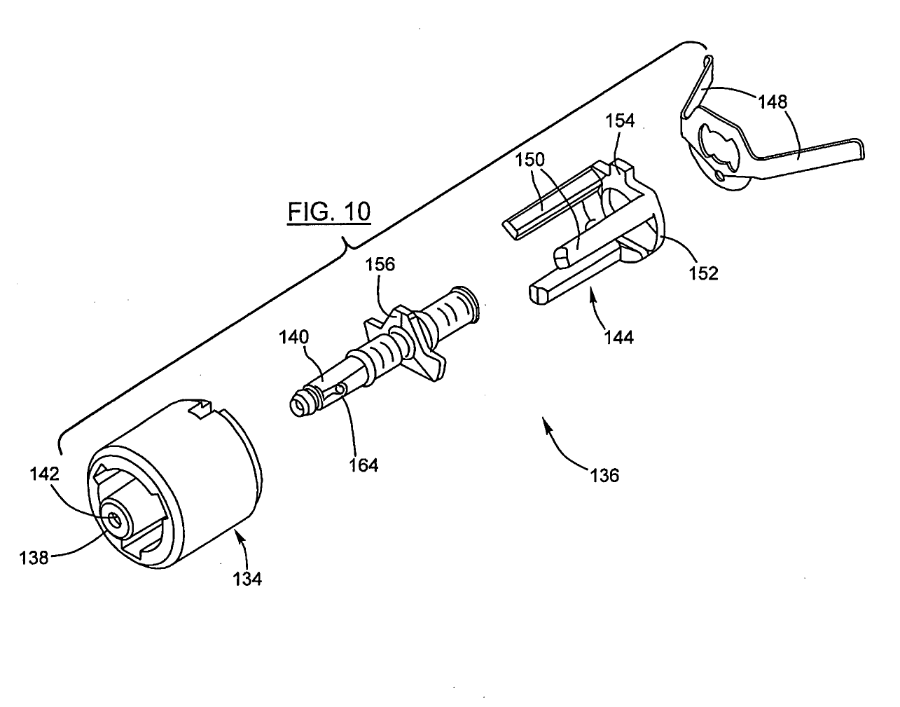

As best shown in FIG. 10, the second syringe outlet port 86 includes a male Luer insert 134, an outlet valve means generally shown at 136 for opening and closing the access to the fluid channel 92 to control the flow of the blood sample therethrough. The male Luer insert 134 includes an opening 138 and a thread for the LUER fitting for coupling with female Luer 31 of a needle 32. The outlet valve means 136 includes a valve element portion 140 and a valve seat portion 142 and first actuating means generally shown at 144 for actuating the valve element portion 140 relative to the valve seat portion 142. A pair of resilient members 148, such as a spring, biases the outlet valve means 136 in a closed position. As will be described, the first actuating means 144 is operable to displace the valve element portion 140 in different directions when the second syringe body portion 84 is either engaged or disengaged with a female Luer 31.

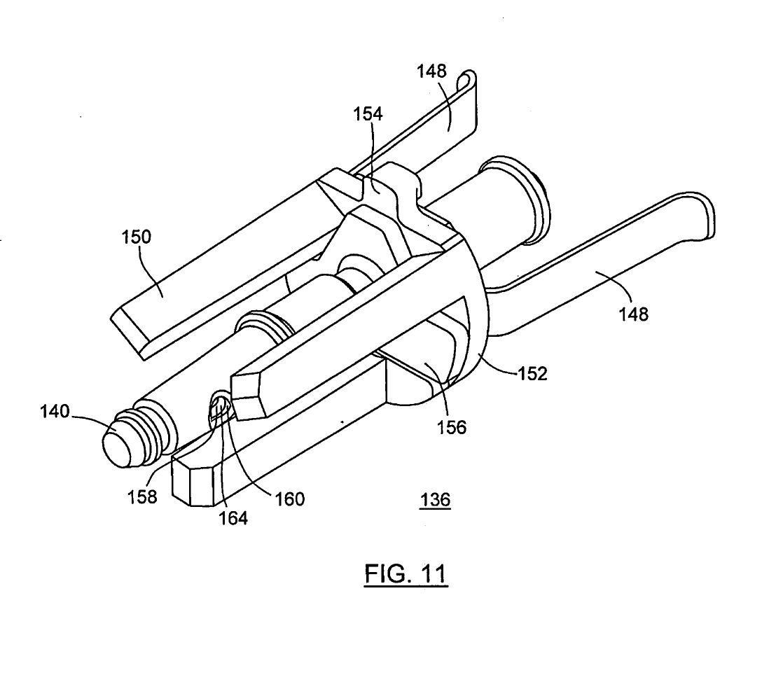

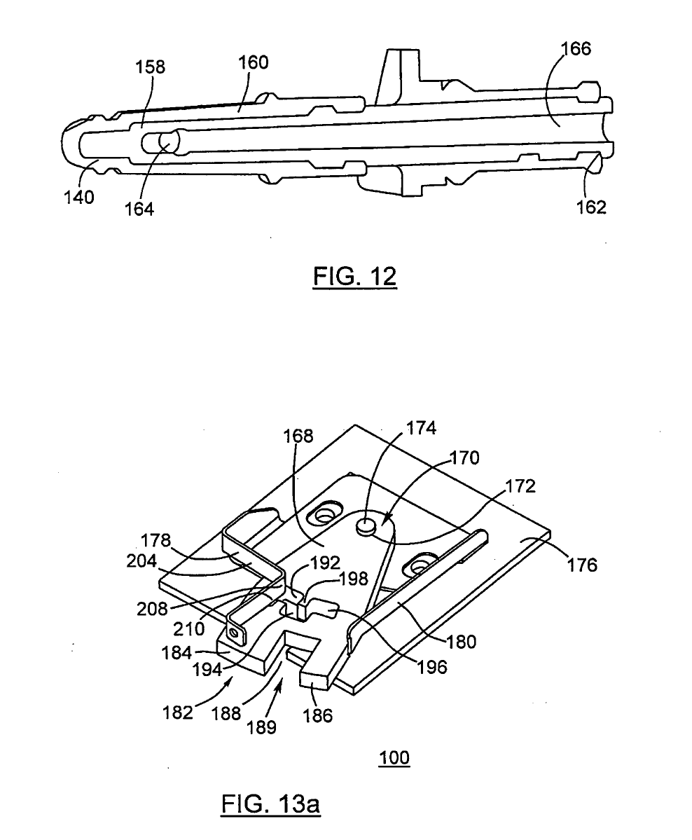

The first actuating means 144 takes the form of a plurality of first actuating elements 150 which extend outwardly from a central web 152, and also second actuating means such as a tab 154 extending therefrom. The central web 152 is fixed to a block 156 positioned in a channel 94 in the body portion 92 of the second syringe 15. The block 156 has a central bore 158 carrying a tubular valve stem 160 having one end carrying the valve element portion 140 and an opposite end carrying a valve stem head 162, which has a peripheral edge region with a sealing element such as an O-ring or the like. The valve stem 160 has a pair of fluid transfer holes as shown at 164 immediately beside the valve element portion 140, thereby forming an inner valve passage which is in fluid communication with the second sample receiving chamber 89, as shown in FIGS. 11 and 12. The female Luer 31 includes complementary first actuating elements which displace the first actuating elements 150, when the female Luer 31 member is introduced into the male Luer insert 134. Consequently, the valve stem 160 and the valve element portion 140 are caused to open the central bore 158 within the valve stem 160 to the channel 96 to allow fluid flow through the outlet port 86.

The outlet port 86 of the second syringe 15 is operable between three states, a locked state, an open state and permanently locked state, by a releasable lock means, such as locking mechanism 100, as shown in FIGS. 13(a) to 13(d). The locking mechanism 100 includes a pawl 168 coupled to the outlet valve means 136 to control the coupling of the female Luer 31 to the male Luer insert 134 of the second syringe 15. The pawl 168 has one end 170 with an opening 172 for receiving a pivoting pin 174, protruding from a board 176, to allow pivoting thereabout. The pawl 168 is positioned between a first spring plate 178 and a second spring plate 180 which control its swinging motion. Typically, the first spring plate 178 is made from fuse material which temporarily changes consistency under the presence of the predetermined electric current signal, such as nickel titanium naval ordinance laboratory intermetallic material (NITINOL). Nitinol exhibits superelasticity and shape memory, such that nitinol is caused to heat up due to the predetermined electric current signal, as such it is mechanically deformed under stress above a specific temperature, and returns to the pre-stressed position when the stress is removed.

On the other end 182 of the pawl 168 is a first finger 184 and a second finger 186 defining a recess 188 with an opening 189. Adjacent to the recess 188 is a punched out slot 190 which includes a plurality of interconnected slots 192, 194, 196. These interconnected slots 192, 194, 196 correspond to the above-mentioned locked state, the open state and the permanently locked state, respectively. The slots 192 and 196 are opposite each other and separated by a pawl tooth 198 on one side of slot 190 and linked to one another by slot 194 on the other side of slot 190. The slot 192 is L-shaped and includes one arm 200 and another arm 202 which links to slot 194.

The first spring plate 178 is secured to the board 176 at one end and includes an arcuate portion 204 positioned above the pawl 168. The arcuate portion 204 is bent at approximately 90 degrees at point 208, and adjacent to the point 208 is an abutment flange 210 which engages the arm 200 of slot 192, in the locked position, as shown in FIG. 13(a). The subsequent positioning of the abutment flange 210 determines the operating state of the syringe 15.

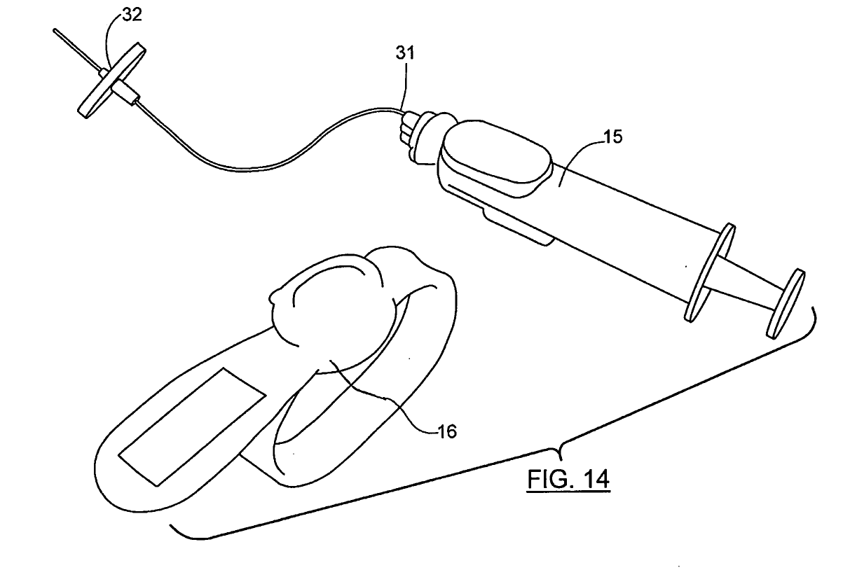

The motion of the pawl 168 through the three different positions will now be described. Starting in the rest position, the abutment flange 210 is positioned in the arm 200 of slot 192. Upon receipt of the release signal following the verification process, a predetermined electric signal is caused to flow through the first spring plate 178, and the electric signal is sufficient to cause the first spring plate 178 to relax. The first spring plate 178 is sufficiently relaxed such that the second spring plate 180 forces the abutment flange 210 out of the arm 200 into arm 202, and finally into slot 194 corresponding to the open position, as shown in FIG. 13(b). A female Luer 31 of a needle 32 can now be attached to the second syringe 15 and the treated blood is expressed from the second sample receiving chamber 89 via the open outlet valve into the patient 17, as shown in FIG. 14.

After a predetermined time, such as 20 minutes, the predetermined electric signal is once again caused to flow through the first spring plate 178, and causes the first spring plate 178 to relax. The second spring plate 180 forces the abutment flange 210 out of the slot 194 into slot 196 corresponding to the permanently locked position, as shown in FIGS. 13(c) and 13(d). If the female Luer 31 is still attached when the release signal is issued, then the abutment flange 210 is prevented from sliding into the permanently locked position until the female Luer 31 is removed. By permanently locking the second syringe 15, subsequent use of the second syringe 15 is precluded, thus substantially eliminating contamination risks.

The operation of the outlet valve means 136 in conjunction with the locking mechanism 100 will now be described with particular reference to FIGS. 10-14. In the locked position of the second syringe 15, the tab 154 rests on the finger 184 and thus restricts the central web 152 from longitudinal displacement away from the opening 138. Any attempt to couple a female Luer 31 fails, since the complementary first actuating elements cannot displace the first actuating elements 150 and therefore the female Luer 31 and male Luer insert 134 cannot mate. Correspondingly, the outlet valve means 136 is biased closed by the pair of resilient members 148 acting on the central web 152, and thus the central bore 158 within the valve stem 160 is closed.

Upon energising the first spring plate 178, the pawl 168 is caused to rotate in a clockwise direction and the abutment flange 210 is forced out of the arm 200 into arm 202, and slides into slot 194 corresponding to the unlocked or open position. Concurrently, the finger 184 of the pawl 168 moves away from the tab 154 such that the tab 154 is now aligned with the recess 188. The female Luer 31 is now be introduced into the male Luer insert 134, and the complementary first actuating elements abut the first actuating elements 150. The force applied to mate the female Luer 31 to the male Luer insert 134 displaces the first actuating elements 150 away from the opening 138, the central web 152 moves in sympathy. The tab 154 enters the recess 188 via the opening 189 and travels the length of the recess 188. The force applied to couple the Luers 31 and 134 is sufficient to compress the resilient members 148 and thus open the central bore 158 within the valve stem 160.

As the treated blood often includes bubbles of gases used during treatment, the second syringe 15 includes a de-bubbling system or bubble removal mechanism to expel gas from syringe, before the treated blood sample is administered to the originating patient 17. Alternatively, a separate vent cap is attached to the proximal end 84 to interface with the Luer 134. The vent cap includes a hydrophobic gas permeable membrane to prevent blood from escaping. Generally, more air can be introduced into the second sample receiving chamber 89 to coalesce the existing bubbles, thus facilitating removal of otherwise small bubbles. Thus, the barrel 83 is transparent such that a user can inspect the treated blood sample to verify that gas bubbles have been removed.

After the treated blood has been administered to the patient 17, the female Luer 31 is uncoupled from the male Luer insert 134, as the needle 32 is removed. With the complementary first actuating elements removed from the male Luer insert 134, the resilient members 148 expand to push the central web 152 towards the opening 138 and the tab 154 travels out of the recess 188 and faces the recess opening 189. At the predetermined time, a predetermined electric signal is caused to flow through the first spring plate 178, and the abutment flange 210 is forced out of the slot 194 into slot 196. The tab 154 now abuts the finger 186, and thus any longitudinal displacement of the central web 152 from away from the opening 138 is precluded. With the abutment flange 210 unable to be forced to return to slot 194, the second syringe 15 is now permanently locked, and so a female Luer 31 can not be subsequently coupled to the male Luer insert 134, as shown in FIG. 13(d).

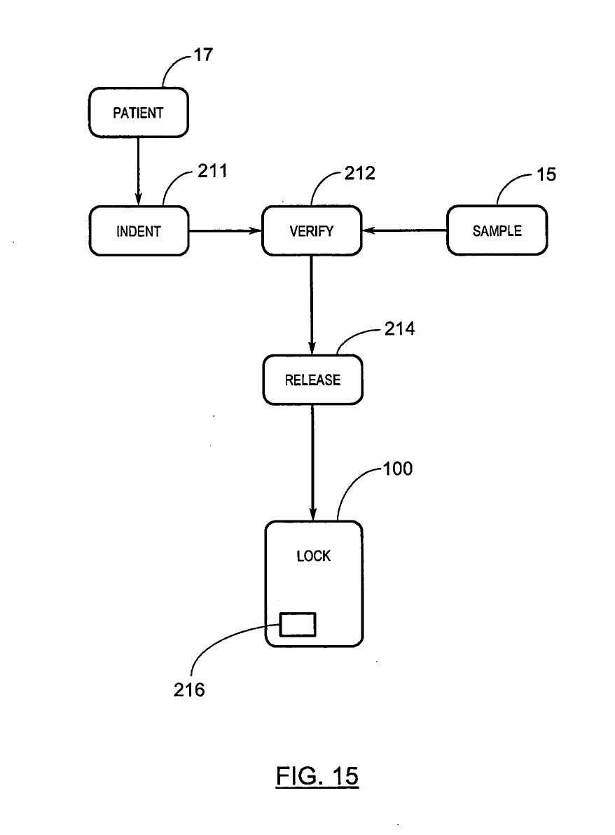

As will be described, the system 10 provides a verification protocol which involves number of verification checks to be sure that the proper treated blood sample is delivered to the proper originating patient 17, and that certain events in the collection, treatment and delivery of the blood sample to the patient 17 occurs within prescribed time periods. To that end, and as shown in FIG. 15, the system has identification means 211 for identifying an originating patient 17, and the untreated blood sample in the first syringe 11, verification means 212 for verifying a match between the originating patient 17 and the treated blood sample in second syringe 15, and release signal generating means 214 for generating a release signal in response to a positive outcome by the verification means. The release signal is transmitted to the releasable lock means 100 to deliver the predetermined current to the first spring plate 178, thereby to render the second syringe 15 operable to deliver the treated blood sample to the originating patient 17.

As will be described, the identification means 211 and the release signal generating means 214 are located on the second syringe 15, but may be located in the aforementioned entities. The releasable lock means 100 has a signal receiving means 216 for receiving the release signal.

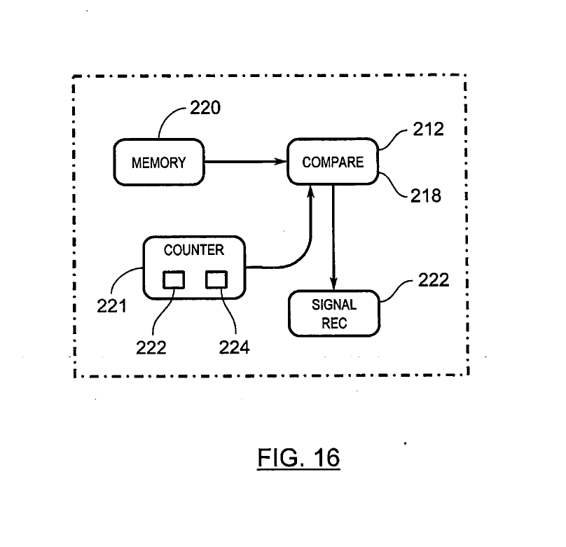

As shown in FIG. 16, the verification means 212 includes comparison means 218 for comparing patient identity data with treated blood sample identity data, both stored in memory means 220, and signal receiving means 216 to receive one or more signals associated with the originating patient identity data and/or the blood sample identity data (either untreated, treated or both). In this case, the one or more signals contain the originating patient identity data and/or the blood sample identity data. However, as an alternative, the one or more signals may contain data which is associated with or related to the patient 17 or blood sample identity data. For example, the data in the signals may include one or more codes which allow the patient identity data or the blood sample identify data to be obtained from a data structure in the memory means 220 or some other location, for example in the form of a look up table, for instance

The verification means 212 also includes a counter means 221 which provides temporal data related to a predetermined event including and/or between an untreated blood sample collection event and a treated blood sample delivery event. The temporal data may also include at least one elapsed time value between two predetermined events including or between the untreated blood sample collection event and the treated blood sample delivery event. The counter means 221 may be implemented as a first incremental counter 222 on first syringe 11 and a second incremental counter 224 on the second syringe 15 are used to track time delay. The first incremental counter 222 tracks the events related to the collection of untreated blood, while the treatment and post treatment events are tracked by the second incremental counter 224. These two incremental counters 222 and 224 operate independently of one another and do not require to be synchronized with each other. The battery power is sufficient to maintain substantial accuracy of their internal clock within the time period from collection of the untreated blood sample to the delivery of the treated blood sample to the patient 17. Therefore, the possibility of losing time or decreasing clock accuracy as the battery's power runs down is substantially eliminated.

In this case, the verification means 212 may be operable to prevent release of the locked third fluid coupling when the elapsed time value has exceeded a predetermined value. Before treatment of the untreated blood sample, the verification means 212 is also operable to prevent treatment of the blood sample when the elapsed time value has exceeded a predetermined value. Similarly, following treatment, the verification means 212 is operable to verify a match between the untreated blood sample in the first syringe 11 and the originating patient 17.

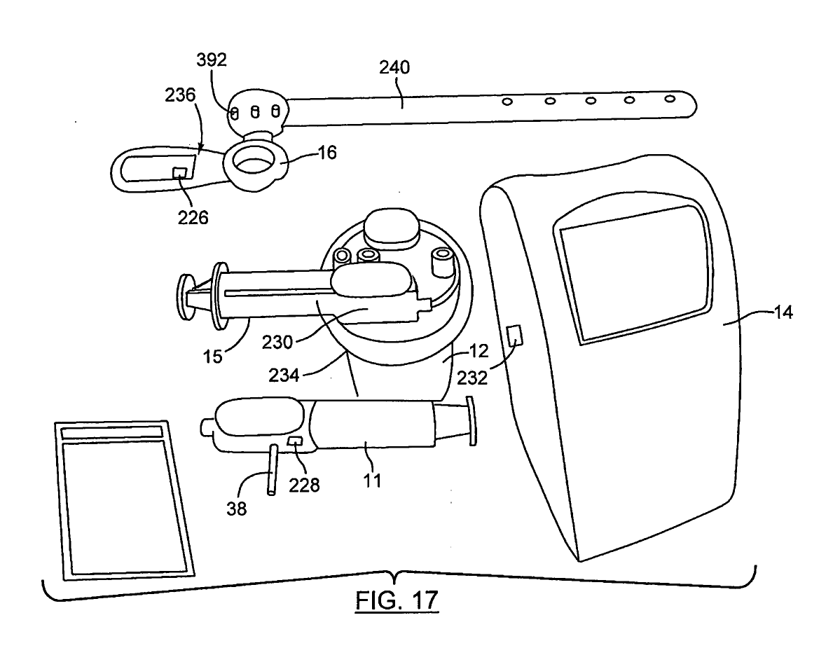

The verification protocol may be implemented in a number of forms, although the most preferred at present is by the use of one or more radio frequency signal transmitters and receivers and RFID tags. As shown in FIG. 17, the wristband 16 is provided with a passive RFID tag, such as WB RFID tag 226, while the first syringe 11 and the second syringe 15 include the aforementioned printed circuit board (PCB) 102 having circuitry for transmitting, receiving and storing data related to the syringe and/or its contents or the originating patient 17, including a S1 RFID reader/writer 228 and a S2 RFID reader/writer 230, respectively. The passive WB RFID tag 226 comprises an antenna coil and a silicon chip that includes modulation circuitry and non-volatile memory. The passive WB RFID tag 226 is energized by an external time-varying electromagnetic radio frequency (RF) wave that is transmitted by a RFID reader/writer, such as the S1 RFID reader/writer 228 or the S2 RFID reader/writer 230. Therefore, S1 RFID reader/writer 228 or the S2 RFID reader/writer 230 is capable of writing data onto the WB RFID tag 226, and reading data back from WB RFID tag 226 by detecting the backscatter modulation.

The blood treatment unit 14 is also equipped with a BTU RFID reader/writer 232 to receive a pre-treatment identity data from the S1 RFID reader/writer 228 and to receive post treatment data from the S2 RFID reader/writer 230. Similarly, the blood treatment chamber 12 is equipped with a passive SMU RFID tag 234 to provide an identification code. The BTU RFID reader/writer 232 issues query signals to the SMU RFID tag 234 to determine whether the blood treatment chamber 12 is valid for use in the treatment process, that is, whether the blood treatment chamber 12 is an authentic product or whether it has been previously used.

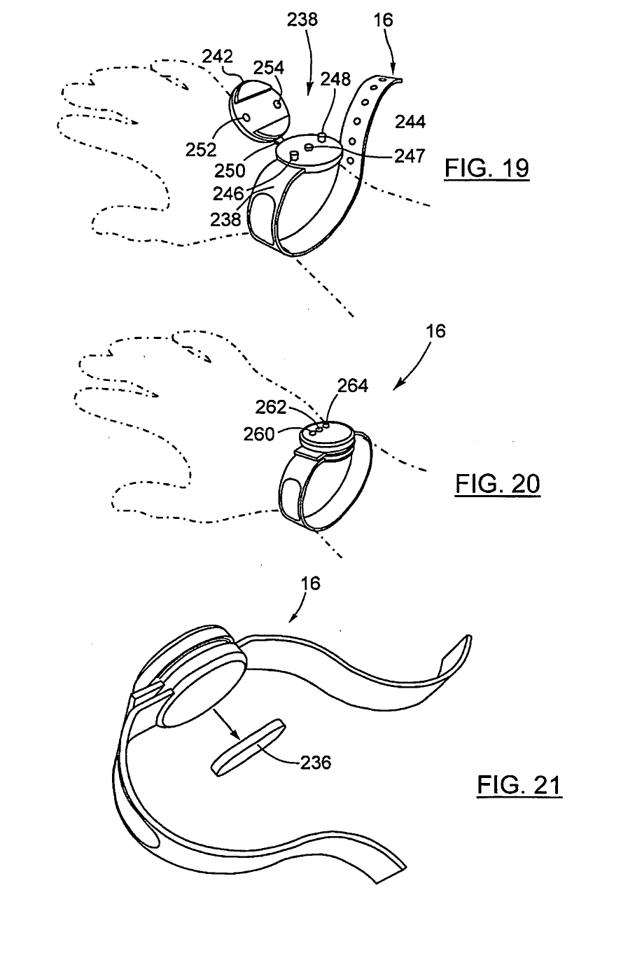

As shown in FIGS. 19 to 21, the wristband 16 (WB) contains a removable portion 236 containing the WB RFID tag 226 and audit data written onto it relating to the patient 17 and/or the treated blood sample. The wristband 16 may also include a buckle assembly 238 having a base portion 240 and cover portion 241. The base portion 240 is integrally formed with a band 242 of resilient material which a number of perforations forming passages 244 to receive the buckle assembly 238. The base portion 240 has pins 246, 247, 248 that are dimensioned to fit through the passages 244. The cover portion 242 is hinged to the base portion 240 by way of a hinge shown at 250. The cover portion 242 also has a pair of cavities 252, each for receiving one of the pins 246 or 248. The pin 247 may press against a switch (not shown) in the base portion 240 to activate portions of the circuitry of the wristband 16, upon securement of the band 242 around the patient's 17 arm.

The method of monitoring a material sample will now be described with reference to the FIGS. 1 to 21. The verification protocol makes use of a number of identification codes, such as a first syringe identity code representative of the untreated blood sample therein, and the a wristband identity code representative of the originating patient 17. To simplify the data transfer, the first syringe identity code and the wristband identity code may include common identity data, though the data between them may be different or related as the case may be. The first syringe identity code may, if desired, include a first time value representative of the time of untreated sample collection from the originating patient 17 (or a designated event either before or after the sample collection step) and/or verification thereof. Thus, the S1 RFID reader/writer 228 functions as a first signal emitter for emitting a first signal carrying the first syringe identification code data, and/or common identity data, while the WB RFID tag 226 on the wristband 16 functions as a first signal receiver to receive the first signal. The second syringe 15 is assigned a second syringe identity code, which is representative of the treated blood sample therein. The second syringe identity code includes a second time value representative of the time of the treated sample delivery thereto from the treatment cavity 52 (or a designated event either before or after the treated sample delivery step) and/or verification thereof.

Thus, the S2 RFID reader/writer 230 functions as a second signal emitter for emitting a second signal carrying the treated blood sample identity data and the WB RFID tag 226 functions as a second signal receiver means to receive the second signal, wherein the verification means 212 is operable to compare the first signal data with data representative of the treated blood sample.

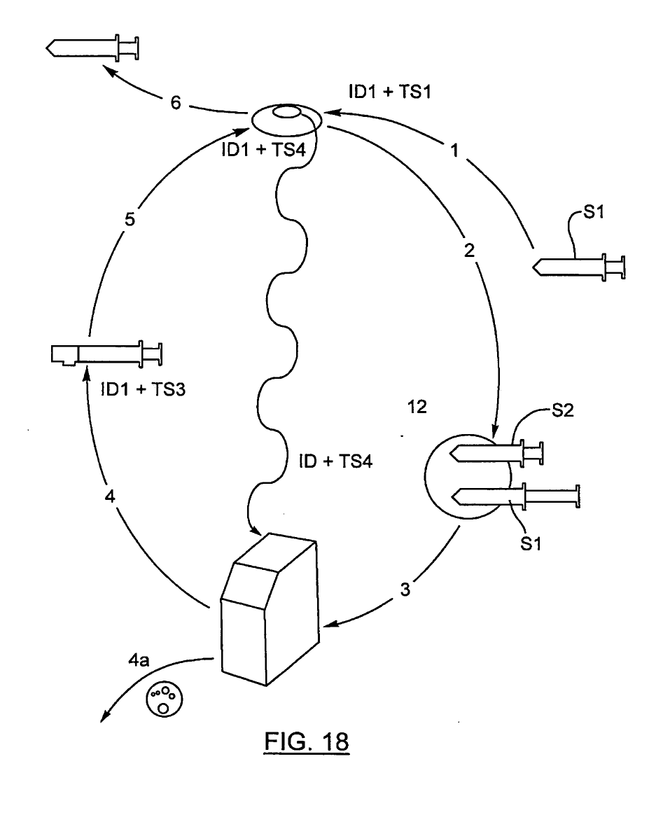

Referring to FIG. 18, the verification protocol will now be discussed along with a typical blood treatment procedure. As shown in FIG. 1, a kit for a blood treatment procedure is assembled including, among other things, the wristband 16, the first syringe 11, the second syringe 15, the sample management unit 12 and a number of prepared labels 258 with patient identification printed thereon. The procedure starts with the activation of the first syringe 11 via an actuating means such as the pullout tab button 38. Once activated, the circuitry on PCB 34 is powered on by the batteries 48 and conducts a power-on-self-test (POST) procedure and subsequently the first syringe 11 is ready for use, barring any detected faults during the POST procedure. The S1 RFID reader/writer 228 is then activated and starts transmitting query signals and waits for an acknowledgement response from the passive WB RFID tag 226. The first incremental counter 222 is also initiated and outputs temporal data, and keeps track of the untreated blood events and log time stamps associated with predefined untreated blood events, in association with the logic means. To that end, a timestamp TS0 indicative of the event of power-on is recorded by the second incremental counter 224 and stored in memory. The S1 RFID reader/writer 228 and the WB RFID tag 226 each contain common patient identity data or sample treatment data, coded as ID 1.

Before the first syringe 11 is used to draw blood from the patient 17, a blood anti-coagulant, such as sodium citrate solution, is also drawn into the first sample receiving chamber 21 to prevent clotting of the blood, as shown in FIG. 3. A sample of blood is then withdrawn from the patient 17, and once primed, the first syringe 11 is brought to within RF range of the wristband 16. The S1 RFID reader/writer 228 queries the WB RFID tag 226 to verify that the data read from or emitted by the WB RFID tag 226 corresponds to the common patient identity data ID1 on S1 RFID reader/writer 228. The process is terminated if there is no correlation between the data on the wristband 16 and the first syringe 11. However, if a positive correlation has been made, the S1 RFID reader/writer 228 records a “time data stamp” TS1 stamp on the S1 RFID reader/writer 228, and writes the same time-stamp to the WB RFID tag 226, Therefore the S1 RFID reader/writer 228 and the WB RFID tag 226 now carry TS0, TS1 and ID1. As an example, the data now on the S1 RFID reader/writer 228 and the WB RFID tag 226, the may be represented as: S1 ID1 TS0 TS1 meaning that the untreated blood sample drawn into the first syringe 11 is from a patient with the identification ID1, the first syringe 11 was powered on at time TS0, and the common patient identity data ID1 on the first syringe 11 and the wristband 16 was matched at time TS1.

The first syringe 11 logic means receives temporal data from the first incremental counter 222 and determines the elapsed time from the start of the procedure (TS0) and the instant that the common patient identity data ID1 on the first syringe 11 and the wristband 16 is matched. The process advances as long as the time unit difference between TS0 and TS1 is within an acceptable predefined range.

In the next step, the first syringe 11 is installed on the blood treatment chamber 12 (with the second syringe 15 already positioned thereon), which is then delivered to the blood treatment unit 14. As such, the S1 RFID reader/writer 228 emits the data TS0, TS1, ID1 to the BTU RFID reader/writer 232. The data also include a time value TS2 denoting a treatment start time. The blood treatment unit 14 then calculates the time delay between TS1 and TS2 of the first syringe 11. In addition, the blood treatment unit 14 issues a query signal to the SMU RFID tag 234 on the sample management unit 12 and, in response thereto, the SMU RFID tag 234 issues a signal containing its identification code to the blood treatment unit 14. A determination as to whether the SMU RFID tag 234 is valid, and also whether the delay is acceptable. If the SMU RFID tag 234 is invalid, and/or the delay is unacceptable then the process ends, otherwise the process continues. This identification code, in this case, includes an “enable” code indicating that the blood treatment chamber 12 has not been previously used for a blood treatment, thus reducing the risk of contamination the current untreated blood sample SI. Alternatively, the SMU RFID tag 234 need not issue an enable code, but rather merely emit a signal containing identity data such as a SKU or the like.

If the time delay between TS1 and TS2 is acceptable, the blood treatment unit 14 the procedure continues with the untreated blood sample in the first syringe 11 being delivered to the treatment cavity 52, via the chamber inlet 50 and conduit 128. The S1 RFID reader/writer 228 is subsequently disabled to prevent further use by including a disable code thereon. In addition, a SMU RFID tag 234 on the blood treatment chamber 12 receives a disable code from the BTU 14 after the blood sample is delivered to it, thereby preventing the reuse of the blood treatment chamber 12. Alternatively, the SMU RFID tag 234 may be disabled in other ways without writing a disable code thereon. For example, the SMU RFID tag 234 may be rendered inoperable by issuing the SMU RFID tag 234 a signal causing a fuse to be blown therein.

In the course of the treatment, the second syringe 15 is powered on and starts querying the BTU RFID reader/writer 232 for data. A new time stamp signifying the end of the blood sample treatment “TS3” is written to the BTU RFID reader/writer 232, and subsequently TS3 is read by the S2 RFID reader/writer 230, and stored thereon. The treated blood is then delivered from the treatment cavity 52 via the conduit 130 and to the second syringe 15, and. If desired, the blood treatment unit 14 may also include the TS1 stamp, meaning that the data written to the S2 RFID reader/writer 230 would include ID1, TS0, TS1, TS2, and TS3. In this case, the second syringe 15 includes the treatment start time TS2 and the treatment end time TS3. Alternatively, or in addition, TS2 or TS3 may include a treatment duration time, or some other code indicating that all previous verification steps have been successfully carried out.

For example, the blood treatment unit 14 may record the following data:
S1 ID1 TS0 TS1
PATIENT ID
TREATMENT START TS2
TREATMENT END TS3
S1 ID1 TS0 TS1 TS3

In this case, the PATIENT ID code may include other patient-related data that is manually or automatically entered into the blood treatment unit 14. Alternatively, the patient-related data is transferred to the blood treatment unit 14 from a central data storage centre, a server computer, a memory bank or the like.

The second syringe 15 is then transported back to the originating patient 17 wearing the wristband 16 and the S2 RFID reader/writer 230 continually polls the WB RFID tag 226 until the latter is within range of the query signals. In response to the query signals, the WB RFID tag 226 then emits ID1 data, at time “TS4”. The S2 RFID reader/writer 230 then calculates the time delay between TS3 data and the time of arrival, TS4, of the second syringe 15 back to the wristband 16. If the expected time delay is exceeded, then the second syringe 15 remains locked by the locking mechanism 100, otherwise the process continues.

The second syringe 15 records ID1, and the time stamp “TS4”. In addition, the second syringe 15 may include the PATIENT ID data as well as the ID1, TS1, TS2, TS3. This data is subsequently written onto the WB RFID 226. At this stage, the S2 RFID reader/writer 230 issues a release signal to the locking mechanism 100 to unlock the second syringe 15, by issuing a predetermined current to the spring plate 178 to force the abutment flange into slot 194, thereby rendering the second syringe 15 operable for injection.

As an example, the WB RFID tag 226 therefore records:
S1 ID1 TS0 TS1
S2 15 ID1 TS0 TS1 TS2
SAMPLE MATCH TS3
S2 UNLOCK TS4

The verification protocol is then completed when the TS4 is recorded in the WB RFID tag 226 after it performs a sample match between the ID1 data on the S2 RFID reader/writer 230 and the WB RFID tag 226. As shown in FIG. 21, the removable portion 236 of the wristband 16 is then separated therefrom and matched with the originating patient's record and the patient record is returned to the blood treatment unit 14 for a data exchange between the WB RFID tag 226 and the blood treatment unit 14, to complete the audit trail.

Alternatively, an RF reading audit record capture station may be provided which is local to the patient 17 or to a patient record area in a medical facility, thereby eliminating the need for the patient record to be returned to the blood treatment unit 14. In this case, the audit record capture station may be capable of downloading the patient record to complete the audit trail. The RF reading audit record capture station may be part of the internal network of the medical facility, either through a wired or wireless data port, or may be part of a network localized to one or more blood treatment unit systems in the medical facility. It may collect data and allow for later batch recording to a computer readable medium, such as an optical disc, hard drive or other storage device. It may be attached to or integrally formed with a computing device, personal digital assistant, a mobile phone or the like. It may also be embodied as software configured to run on a computing device, together with an RFID reading attachment thereon.

The data ID1 and TS4 is delivered to blood treatment unit 14 or other system to complete the audit trail. The time stamp may also include an “event” code, which may comprise five major events:
1) S1 start time
2) WB acknowledges with S1
3) Start of Treatment
4) End of Treatment
5) Match between the Treated Sample and the Originating Patient.

The time stamp may also include any one or more of a number of Error events
1) No match
2) S1 does not match with WB at before/after collection
3) S2 does not match with WB on return after Treatment.
4) Time Delay-exceed time to collect of blood
5) Time Delay-exceed time to deliver sample to BTU
6) Time Delay-exceeds time to return to patient.

The TS3 time stamp may also include a “match” code as follows:
01 Match
02 No match

In another embodiment, the wristband includes electronic circuitry coupled to the passive WB RFID tag 226, and a battery for providing power to the electronic circuitry. As shown in FIG. 20, the wristband 16 includes outputs means, such as LEDs 260, 262, 264, or a speaker (not shown), which are operated in different combinations of one or more thereof. For example, the LEDs 260, 262 may be operable to illuminate in accordance to a predetermined cycle indicative of the communication associated with verification process with the first syringe 11 and the second syringe 15. The third LED 264 may be provided for alarm situations.

In another embodiment, the wristband includes electronics circuitry coupled to the passive WB RFID tag 226, and a battery for providing power to the electronic circuitry. As shown in FIG. 20, the wristband 16 includes outputs means, such as LEDs 260, 262, 264, or a speaker (not shown), which are operated in different combinations of one or more thereof. For example, the LEDs 260, 262 may be operable to illuminate in accordance to a predetermined cycle indicative of the communication associated with verification process with the first syringe 11 and the second syringe 15. The third LED 264 may be provided for alarm situations.

The wristband 16 may be replaced by some other article to be worn, carried, attached or ingested by the patient 17, such as a pinned or self adhesive label 258 and the like.

The second syringe 15 may also include a second sample receiving chamber 89 volume detector to determine whether the received treated blood from the treatment cavity 52 is within a predefined range suitable for injection into the patient 17 to provide the desired medical treatment.

In another embodiment, the system 10 includes a blood sample treatment chamber, similar to the sample blood treatment chamber 12 of FIG. 4, with an expandable treatment cavity 52 formed by a cover portion 54, a bottom portion 56 and a flexible walled portion 58 therebetween.

In yet another embodiment, the system includes a locking mechanism 100 operable by a solenoid or motorized means configured to receive the release signal.

In another embodiment, the system includes a wristband 16 with electronic circuitry for transmitting, receiving and storing data related the originating patient 17, such as identification data or an identifier, SKU, serial no., manufacturing date, expiry date, health facility data, health practitioner data, medication data, and so forth. The circuitry includes, but is not limited to, a transmitter, a receiver, logic means or processor, a computer readable memory for data storage, a timing circuit, an antenna and a power source. The circuitry also includes an RFID reader/writer for reading RFID tags associated with other entities within the treatment system, such as the first syringe 11, the second syringe 15, or the sample management unit 12. A wristband tag. This wristband 16 acts as the archive data storage for the entire treatment and therefore provides the audit trail once the treatment has been completed. The data may be stored in the computer readable medium, such as RAM, ROM, flash memory, and so forth. or the wristband may include an RFID tag to which the data is written.

The first syringe 11 and the second syringe 15 include a printed circuit board (PCB) having circuitry for transmitting, receiving and storing data related to the syringe and/or its contents or the originating patient 17, such as identification data or identifiers, SKU, serial no., manufacturing date, expiry date, fluid data, health facility data, health practitioner data, medication data, and so forth. The circuitry is implemented as an active RFID tag having a transmitter, a receiver, logic means or processor, a computer readable medium for data storage, a timing circuit, an antenna and a power source such as a batteries. Also coupled to the PCB are input/output devices such as a display, LED, a speaker or a button. The second syringe 15 also includes circuitry for controlling the operation of a releasable lock means or electromechanical interlock to prevent re-injection of treated blood in the event that the wristband 16 identifier and second syringe identifiers do not match.

Similar to the preferred embodiment, the system includes a BTU reader/writer which can communicate (read and write) to RFID tags of the first syringe 11 and the second syringe 15 and to a tag on the sample management unit 12. The first syringe 11 RFID tag stores and record data relating to the patient for example, the time blood was removed for treatment. It will also ensure that the syringe 11 cannot be re-used. The first syringe 11 RFID tag will also include an elapsed time counter and a matching identifier to that contained in the wristband written at the time of manufacture or packaging. The second syringe 15 RFID tag includes similar functions and includes logic and circuitry to drive an electromechanical interlock.

The flow of treatment events are similar to the one described above. Prior to removal of blood, a check is performed to verify that the unique treatment set ID numbers contained in the wristband 16 and in the first syringe 11 match, by having the syringe 11 active tag emit the data to the wristband 16 reader/writer. If there is a match, this event is recorded by the wristband and blood is withdrawn from the patient. At the same time the elapsed time counters in the syringe 11 tag and wristband 16 will start.

The first syringe 11 is then be fitted onto the sample management unit (SMU) 12, which is already fitted with a single-use second syringe 15. The SMU 12 with both syringes 11,15 is then taken to the blood treatment unit (BTU) 14 with a BTU reader/writer. The patient details are entered into the BTU reader at this stage. The blood treatment unit 12 reader/writer will read the first syringe 11 tag and write the details (including the unique ID) to the second syringe 11 tag. The BTU reader/writer will also write a message to the SMU tag to indicate it has been used. The BTU reader/writer will read the elapsed time from the first syringe 11 tag and calculate treatment time details. These are then written to the second syringe 11 tag along with patient ID.

Following the blood treatment, the BTU reader/writer writes the completed treatment time to the second syringe 15 tag. The SMU 12 is removed from BTU. S2 syringe is removed from the SMU 12, and the SMU 12 and S1 syringe are discarded. The second syringe 15 is then presented to the wristband 12 on the patient and, provided the unique IDs match and elapsed time is within set parameters, the second syringe 15 locking mechanism is released and the second syringe 15 can be used to inject the treated blood into the patient. The wristband 12 reader/writer writes the patient data and procedure details to the wristband 12 tag or computer readable medium, for subsequent removal for storage with patient records. The wristband 12 reader/writer is then deactivated and its strap is cut to allow removal from the patient and disposal. A network RFID reader is used to read the encrypted data in the wristband tag memory unit or computer readable medium for transfer to the health facility database on a computer or network.

In another embodiment, the BTU reader/writer or an external reader/writer provides all the verification checks

Even though the description above is in large part focused on the use of system 10 in the treatment of autologous blood samples, it will be understood that the system 10, its components and alternatives thereof, may be used for samples other than blood samples, such as bone marrow or, lymphatic fluids, semen, ova-fluid mixtures, other bodily fluids or other medical fluids which may or may not be “autologous”, for example fluid mixtures perhaps containing a patient's desired solid sample such as from organs, body cells and cell tissue, skin cells and skin samples, spinal cords. The system 10 may also be used for medical testing where it is important to ensure that test results of a particular test can be delivered to the originating patient 17.

While the system 10 makes use of syringes 11 and 15, it will be understood that other devices may be used such as, alone or in combination, one or more syringes, IV bottles, powder and/or atomized fluids and/or gas inhalant dispensers, implant delivery dispensers, ventilators, syringe pumps, intubation tubes, gastrointestinal feeding tubes, or a plurality and/or a combination thereof. One of the treatment devices may also comprise a blood treatment device such as that disclosed in International Publication No. WO0119318A1 entitled “APPARATUS AND PROCESS FOR CONDITIONING MAMMALIAN BLOOD” (the entire contents of which are incorporated herein by reference). Alternatively, one treatment device may be equipped to perform a range of invasive and non-invasive treatments such as surgeries, treatments for diseases such as cancer, as well as exploratory or diagnostic investigations such as X-rays, CAT Scans, MRI's and the like.

Although the invention has been described with reference to certain specific embodiments, various modifications thereof will be apparent to those skilled in the art without departing from the spirit and scope of the invention as outlined in the claims appended hereto.

1. A system for the collection, treatment and delivery of a blood sample, the system comprising:
an article for association with a patient having a patient identifier;
a first syringe having:
a first syringe inlet for drawing an untreated blood sample from the patient,
a first fluid chamber for receiving the untreated blood,
a first syringe outlet for dispensing the untreated blood sample from the first chamber,
a first incremental counter for recording temporal data corresponding to untreated blood events related to the collection of blood,
the first syringe being associated with a first unique identifier correlatable to the patient identifier;
a vessel for processing the blood sample, the vessel having:
a blood sample processing chamber, the vessel having a chamber inlet; the first syringe outlet being operable to establish a dedicated first fluid coupling with the chamber inlet to dispense the untreated blood sample to the blood sample processing chamber; the vessel having a chamber outlet for dispensing a treated blood sample following treatment to a second syringe,
the second syringe having:
a second syringe inlet operable to form a dedicated second fluid coupling with the chamber outlet to receive the blood sample from the blood sample treatment chamber;
a second chamber for receiving the treated blood;
a second syringe outlet;
a passage in communication with the second chamber and the second syringe outlet;
a second incremental counter for recording temporal data corresponding to blood treatment events, treated blood events and delivery events; the second incremental counter being operable independently of the first incremental counter and being non-synchronized with the first incremental counter;
the second syringe being associated with a second unique identifier, the second unique identifier operatively associated with the first syringe and correlatable to the first unique identifier;
a releasable lock formed within the passage for operating the second syringe outlet between a plurality of states;
a processor having:
a comparator for comparing the patient identifier to the first unique identifier to determine the correlation between same; and comparing the second unique identifier to the patient identifier to determine the correlation between same, the comparator issuing an output signal;
logic for receiving the output signal and the temporal data to determine time delays between the events and for determining whether the time delays are within predefined ranges;
a release signal generator coupled to the logic for issuing a release signal to the releasable lock;
whereby the release signal is issued upon confirmation of the correlation between the patient identifier and the first unique identifier, and the correlation between the patient identifier and the second unique identifier, and provided that the time delays are within predetermined ranges.

2. The system of claim 1 wherein the first syringe further comprises:
an inlet valve assembly in communication with the first fluid chamber; and
an outlet valve assembly disposed intermediate the first fluid chamber and the inlet valve assembly;
the inlet valve assembly including:
an inlet valve member operable in an open position and a closed position;
a resilient member biasing the inlet valve member to its closed position;
the outlet valve assembly including:
an outlet valve member operable between a closed position and an open position; and
a sealing member; and
an anchoring member engaging the outlet valve assembly to maintain the outlet valve member in a closed position;
whereby with the outlet valve member in the closed position, the inlet valve member is placed into the open position upon compression of the resilient member to allow fluid flow into the chamber;
and the outlet valve member is operable by disengaging the anchoring member and defeating the sealing member when the fluid chamber is primed, while the inlet valve member is in a closed position;
thereby to allow discharge from the fluid chamber.

3. The system of claim 1 wherein the releasable lock being operable in response to a release signal to operate the syringe outlet valve between an open state and a closed state.

4. The system of claim 3 wherein the releasable lock includes:
a pivoted pawl member;
interconnected slots corresponding to the closed state, the open state and the permanently closed state;
a first resilient member having a flange restricted to travel within the interconnected slots, wherein the first resilient member is spring made from a fuse material which temporarily changes consistency under the presence of the release signal, the position of the flange within the interconnected slots dictating the state of the outlet valve.

5. The system of claim 4 wherein the fuse material is nickel titanium naval ordinance laboratory intermetallic material (NITINOL).

6. The system of claim 5 wherein the releasable lock includes a second resilient member to force the flange into a slot corresponding to a permanently closed state.

7. The system of claim 6 including the article, first syringe, the second syringe, the vessel, electronic circuitry for transmitting, receiving and storing data related to the collection, treatment and delivery of the autologous blood.

8. The system of claim 7 wherein the circuitry includes any of the following: a transmitter, a receiver, an antenna, processor, computer readable medium, a timing circuit for maintaining temporal data related to the collection, treatment and delivery of the autologous blood sample, a power source and input/output devices.

9. The system of claim 8 wherein the circuitry of the first syringe and the second syringe includes an active RFID tag deriving power from the power source.

10. The system of claim 9 wherein the article includes an RFID reader/writer in communication with the active RFID tags on the first syringe and the second syringe.

11. The system of claim 10 wherein the article includes the processor, the comparator, the logic and the release signal generator to issue a release signal to the releasable lock upon confirmation of the correlation between the patient identifier and the first unique identifier, and the correlation between the patient identifier and the second unique identifier, and provided that the time delays are within predetermined ranges.

12. The system of claim 8 wherein the circuitry of the article and the vessel include a passive RFID tag.

13. The system of claim 12 wherein the first syringe and the second syringe include an RFID reader/writer in communication with the passive RFID tags on the article and the vessel.

14. The system of claim 13 wherein the second syringe includes the processor, the comparator, the logic and the release signal generator to issue a release signal to the releasable lock upon confirmation of the correlation between the patient identifier and the first unique identifier, and the correlation between the patient identifier and the second unique identifier, and provided that the time delays are within predetermined ranges.

15. The system of claim 14 wherein the vessel further comprising:
a body having:
a top portion, a bottom portion, and a walled portion therebetween;
a cover portion sealing received by a body opening adjacent to the top portion to define the blood sample processing chamber;
the cover portion having a gas inlet port coupled for carrying at least one gas into the blood sample processing chamber to interface with the untreated sample, a gas outlet port coupled for carrying at least one gas from the blood sample processing chamber; a chamber inlet port for releasably coupling the first syringe to supply the untreated sample, and a chamber outlet for releasably coupling the second syringe being for receiving a treated sample; and
a temperature sensor for determining the temperature of the at least one fluid in the treatment cavity.

16. The system of claim 15 wherein the walled portion is rigid.

17. The system of claim 15 wherein the walled portion is flexible.

18. The system of claim 15 wherein at least one of said ports includes a Luer connector for coupling to a complementary Luer connector.

19. The cover of claim 15 wherein at least one of said ports includes a bayonet coupling part for coupling to a complementary bayonet coupling part.

20. The system of claim 11 wherein the vessel further comprising:
a body having:
a top portion, a bottom portion, and a walled portion therebetween;
a cover portion sealing received by a body opening adjacent to the top portion to define the blood sample processing chamber;
the cover portion having a gas inlet port coupled for carrying at least one gas into the blood sample processing chamber to interface with the untreated sample, a gas outlet port coupled for carrying at least one gas from the blood sample processing chamber; a chamber inlet port for releasably coupling the first syringe to supply the untreated sample, and a chamber outlet for releasably coupling the second syringe being for receiving a treated sample; and
a temperature sensor for determining the temperature of the at least one fluid in the treatment cavity.

21. The system of claim 20 wherein the walled portion is rigid.

22. The system of claim 20 wherein the walled portion is flexible.

23. The system of claim 20 wherein at least one of said ports includes a Luer connector for coupling to a complementary Luer connector.

24. The cover of claim 20 wherein at least one of said ports includes a bayonet coupling part for coupling to a complementary bayonet coupling part.

25. A method of monitoring a material sample from a patient, comprising the steps of:
(a) collecting the sample from the patient with a first collection device;
(b) associating the patient with a first signal carrying data representative of the sample;
(c) associating the first collection device with a second signal carrying data representative of the sample;
(d) delivering the sample to a sample treatment chamber;
(e) processing the sample to form a processed sample;
(f) collecting the sample in a second collection device;
(g) associating the second collection device with a third signal carrying data representative of the processed sample;
(h) comparing the data in the first and third signals to link the processed sample with the patient; and thereafter
(i) associating at least one of the steps (a) to (h) with temporal data;
(j) determining at least one time delay using said temporal data to determine whether said at least one of the steps (a) to (h) occurs within an acceptable time limit;
(k) delivering the processed sample to the patient upon a positive outcome from step (h) and step (j); and
(l) assembling an audit record having temporal data collected from step (i), the outcome from step (h) and step (j), and data associated with the sample.
